(19) United States
(12) Patent Application Publication
(10) Pub. No.: US 2001/0036665 A1
(43) Pub. Date: Nov. 1, 2001

(54) METHOD OF PREPARING CRYOGENICALLY PRESERVED ADHERENT CELL CONTAINING PLATE FOR TISSUE CULTURE APPLICATIONS

(76) Inventors: SUSAN M. YOUNG, ALBUQUERQUE, NM (US); CHESTER KING, FREDERICK, MD (US); SHAWN A. MATLOCK, FREDERICK, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/271,430

(22) Filed: Mar. 18, 1999

(51) Int. Cl. C12N 5/00; C12N 5/02

(52) U.S. Cl. 435/374; 435/325

(57) ABSTRACT

A process for applying and storing adherent cells on a tissue culture plate 20 for preparing tissue culture plates for use in tissue culture experiments. In the process, adherent cells are removed from a flask using trypsin after being incubated in a CO₂ Incubator. The cell suspension is created using the adherent cells and a liquid culture medium, which is centrifuged and tested for volume using a hemacytometer. The cell suspension is placed in the wells of a tissue culture plate 20 and then incubated. A cryopreservative is deposited into each well and the tissue culture plate is sealed with an adhesive foil cover and cryogenically preserved using liquid nitrogen. The tissue culture plate 20 is then place into a plastic, waterproof, resealable bag enabling it to be stored at −80° C. for up to 3 months and shipped without damaging the morphological or functional nature of the adherent cells. The tissue culture plate forms part of a cryopreserved cell culture assembly which includes frozen adherent cells attached to the bottom wall surface of a well in the tissue culture plate.

This application is a continuation-in-part of application Ser. No. 09/040,379, filed Mar. 18, 1998, which is herein incorporated by reference.

### BACKGROUND

1. Field of the Invention

This invention relates to well slides and cell cultures, with the intent of improving the method of preparing cell cultures for various tissue culture experiments.

2. Description of Prior Art

Currently, adherent cell lines are grown on a variety of plastic containers such as flasks, Petri dishes, and multi-well tissue culture plates. Once these cell lines have reached confluency, they are treated with trypsin-containing solutions to release them from the plastic surface, counted, and either re-plated or frozen. When these cells are needed for culture, they are thawed and plated. These manipulations are time-consuming and statistically increase the chance for contamination. Numerous assays require the use of adherent cell lines at various stages of confluency and it can be difficult to have multiple plates, dishes or flasks at a particular stage of growth. At the present time, these types of cells are frozen in the non-adherent stage and require growing when thawed. Many adherent cell lines lose their particular characteristics after numerous passages.

### SUMMARY OF THE INVENTION

The present invention is directed to a cryopreserved cell culture assembly including a tissue culture plate having at least one well bounded by a bottom wall surface upon which cells can attach and grow, and a plurality of side walls forming a top rim, the rim defining a well opening. Adherent cells in a frozen state attach to the bottom wall surface. The cells contain an amount of a cryoprotectant which is effective to protect them from damage when stored frozen. In more specific aspects of the invention, the cells are animal cells, insect cells or mammalian cells, and in a still more specific aspect of the invention, the cells are receptive for infection by HIV-1, and even more specifically the receptive cells are HeLa-CD4-LTR-β-gal, HeLa-CD4-CCR5-LTR-β-gal, H9, C8166, Molt-4, Jurkat, CEMX174, HUT 78, or U87.CD4.

The invention is also directed to a method of preparing a cryopreserved cell culture assembly, comprising contacting adherent cells attached to a bottom wall surface of a tissue culture plate with an amount of a cryoprotectant effective to protect said cells from damage when freezing, wherein the tissue culture plate has at least one well bounded by said bottom wall surface upon which cells can attach and grow, and a plurality of side walls forming a top rim, the rim defining a well opening. The tissue culture plate is sealed by placing a cover over said top rim and the adherent cells attached to said bottom wall surface are frozen

The present invention provides plates of adherent cells which have been frozen at desired stages of confluency and which are ready for use in various assays, such as neutralization assays, and for feeder cell cultures, upon thawing. The present invention also relates to methods of preparing and assembling such plates.

### BRIEF DESCRIPTION OF THE DRAWINGS

FIG. 1 is a diagram flow chart of the preparation of the adherent cells and cell plates.

FIG. 2 is a perspective view of a well section of a treated tissue culture plate prior to incubation.

FIG. 3 is a perspective, sectional view of a well of a treated tissue culture plate after incubation.

FIG. 4 shows well plate prior to submersion in liquid nitrogen.

FIG. 5 shows a side view of a well treated with adherent cells after incubation.

FIG. 6 shows a well plate, with an adhesive foil cover, after thawing.

### DETAILED DESCRIPTION

The following reference numerals are used:

02 adherent cells;

04 flask;

06 CO₂ incubator;

08 trypsin;

10 culture medium;

12 cell suspension;

14 centrifuge tube;

16 hemacytometer;

18 microscope;

20 tissue culture plate;

22 well;

24 cryogenic preservative;

26 liquid nitrogen;

28 adhesive foil cover;

30 plastic, waterproof, resealable bag; and

32 supernatant.

This invention preferably utilizes adherent cells 02. The cells can be of any origin, including insect, plant, mammal, etc. It is preferred that these cells adhereto the surface of the tissue culture receptacle. Useful cell lines include: F4, V9, F2, epithelial, fibroblasts, T cells, such as lymphoblastoid cell lines, e.g., HeLa-CD4-LTR-β-gal (MAGI); MAGI-CCR-5. See, e.g., et al., J. Virol., 71:3932-3939, 1997. In addition, T cell lines, lymphoblastoid cell lines, H9, C8166, Molt, Molt-4, CEM, Jurkat, preferably, CEMX174, HUT 78, U87.CD4. See, e.g., Virology, 236:208-212, 1997.

In a preferred embodiment, the present invention relates to a cryopreserved cell culture assembly, comprising: a tissue culture plate having at least one well bounded by a bottom wall surface upon which cells can attach and grow, and a plurality of side walls forming a top rim, said rim defining a well opening; adherent cells attached to said bottom wall surface, wherein said cells contain an amount of a cryoprotectant which is effective to protect them from damage when stored frozen, wherein said cells are in the frozen state.

By the term “tissue culture plate,” it is meant any vessel or receptacle in which cells can be grown. For example, a tissue culture plate can be a flask, a petri dish, a six-well, 12-well, 24-well, or 96-well container. It can be constructed from any suitable material, including, plastics such as polystyrene or PETG. The bottom surface of such plates, e.g., the bottom surface of the well, can be treated to facilitate growth and attachment, i.e., “tissue-culture treated.” Any substrate upon which cells adhere can be used, according to the present invention, including but not limited to, polystyrene, polyamino-treated surfaces (such as polysine, etc.), organic and inorganic membranes, polycarbonate membrane inserts, glass, and treated culture plates, e.g., poly-d-lysine and polycarbonate-treated plates. In addition to plates, tissue culture bottles, carriers, and other such culture vessels can be used.

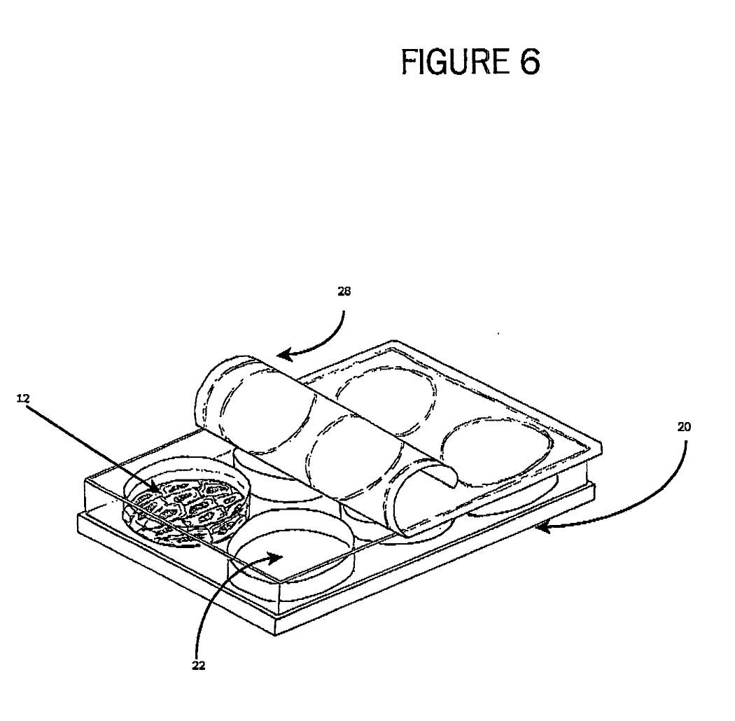

The plate preferably contains at least one-well or receptacle, where the well or receptacle has a bottom surface and a plurality of sides (circular, rectangular, etc.). The sides form at rim at their top which can be raised or unraised. Such plates are commercially available. See, e.g., U.S. Pat. No. 4,012,288 or 5,795,775. Generally, the plate has a cover or lid which can be used to seal the plate around the rims of the well. See, e.g., FIG. 6. Such cover can be attached in any suitable manner, e.g., by adhesive. It can be removable or detachable/

The assembly preferably comprises adherent cells, i.e., cells which stick to the surface of the well. The surface can be treated, as mentioned above, with any agent which facilitates or enhances cell adhesion to it. The adherent cells preferably contain an amount of a cryoprotectant which is effective to protect cells from damage during the freezing process, storage process, or thawing process, e.g., to prevent ice-crystal formation. Useful cryoprotectants include, e.g., dimethylsulfoxide, glycerol, propylene glycol, ethylene glycol, trehalose, raffinose, or hydroxyethyl starch. Effective amounts of cryoprotectants, and methods of freezing, are well-known in the art and can be determined routinely.

The adherent cells 02 are grown in a flask 04 and incubated in a CO₂ incubator 06 at 37° C. with 5% CO₂ levels. The flask 04 is removed from the CO₂ incubator 06 and aspirated, and 5 ml of trypsin 08 are added and allowed to stand for 10 to 15 minutes. Then 5 ml of culture medium 10 are added to the flask 04. Using a pipette, 10 ml are removed from the cell suspension 12. The cell suspension 12 is placed into a 15 ml centrifuge tube 14. The cell suspension 12 is then centrifuged for 7 minutes at 1000×g. The supernatant 32 is aspirated, at which time the cell pellets are located at the bottom of the centrifuge tube 14. The pellets are re-suspended in 10 ml of the culture medium 10. Then, using a Pasteur pipette, one droplet is removed from the cell suspension 12 and placed on a hemacytometer 16 and covered with a slip. The hemacytometer 16 is placed on a microscope 18 to count the cells. Using the cell count, the cell suspension 12 is adjusted to result in a cell count of 4×104 per 1 ml by diluting the cell suspension 12 with a culture medium 10.

A rigid polystyrene tissue culture plate 20 with a plurality of wells 22 is then treated in the following manner. With a pipette, 1 ml of the 4×10⁴ cell suspension 12 is placed in each well 22 of a polystyrene tissue culture plate 20. The entire surface of the bottom of the well is to be uniformly covered with the cell suspension 12; however, when using MAGI CCR-5 (HeLa-CD4-LTR-β-gal) cells, only 30% to 50% of the well 22 bottom is to be covered with the suspension. The tissue culture plate 20 holding the cell suspension 12 is then incubated for 1 hour in the CO₂ incubator 06, with a culture medium containing 30% fetal calf serum, at 37° C. with 5% CO₂, allowing the cells to grow.

After incubation, the tissue culture plate 20 is subjected to a freezing procedure, wherein the temperature is strictly controlled at −160° C., as follows. A 100 μl solution of a cryogenic preservative 24 at 4° C. is pipetted into each well 22 of the tissue culture plate 20. An adhesive foil cover 28 is sealed over the top of the tissue culture plate 20, thus protecting the adherent cells 02, ensuring moveability of the tissue culture plate 20, and permitting movement of the liquid nitrogen 26 around each individual well 22. The tissue culture plate 20 is set into a container holding enough liquid nitrogen 26, at −160° C., to reach above the level of the adherent cells 02 but below the lip of each well 22 in the tissue culture plate 20 for 1 minute. Each tissue culture plate 20 is then placed in a plastic, waterproof, resealable bag 30 and sealed. The tissue culture plate 20 is stored at −80° C. where it can be maintained for 3 months.

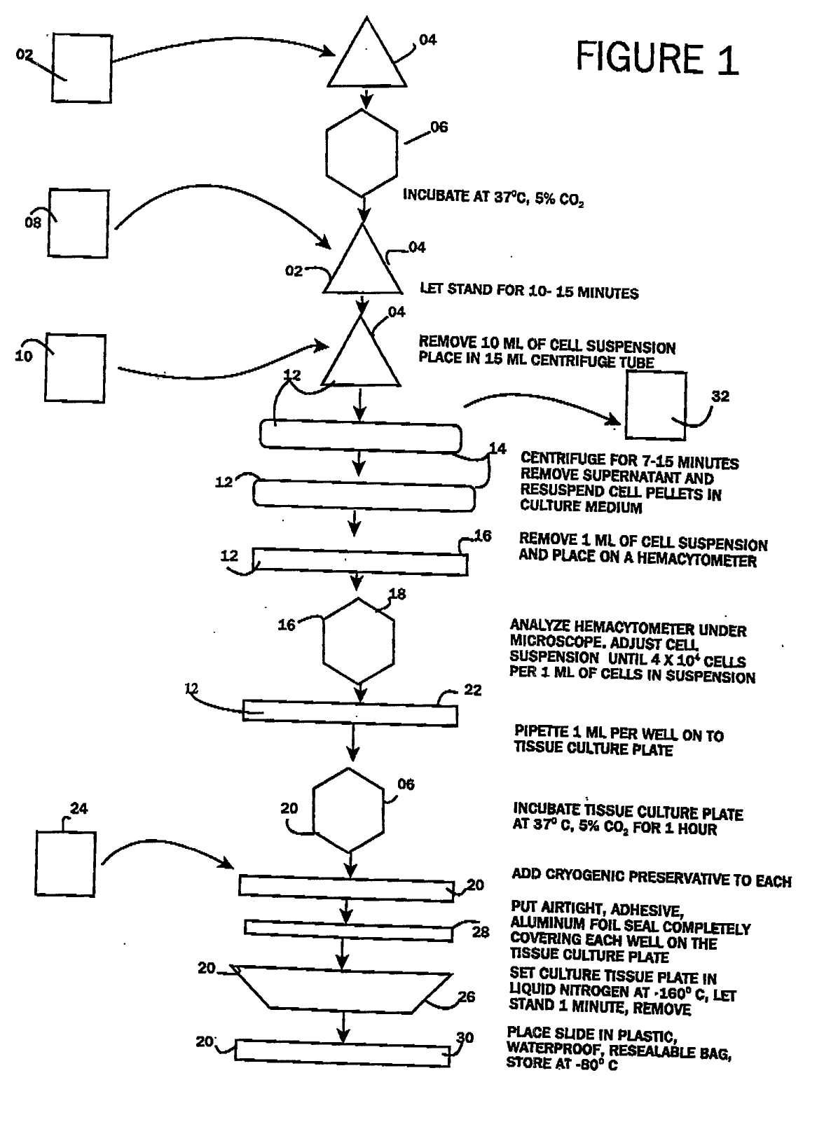

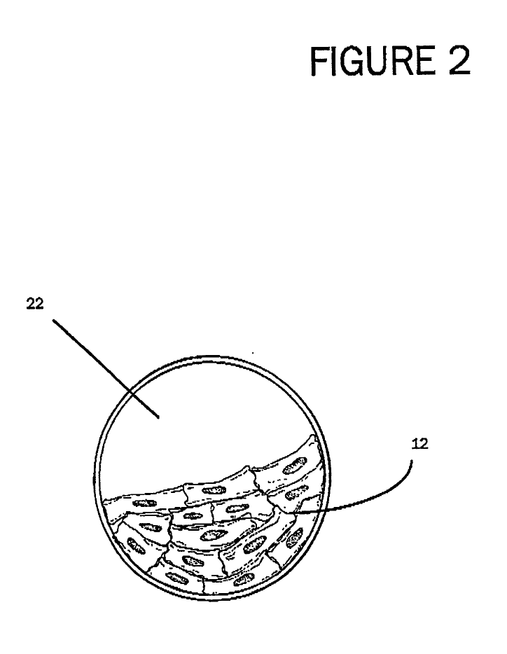

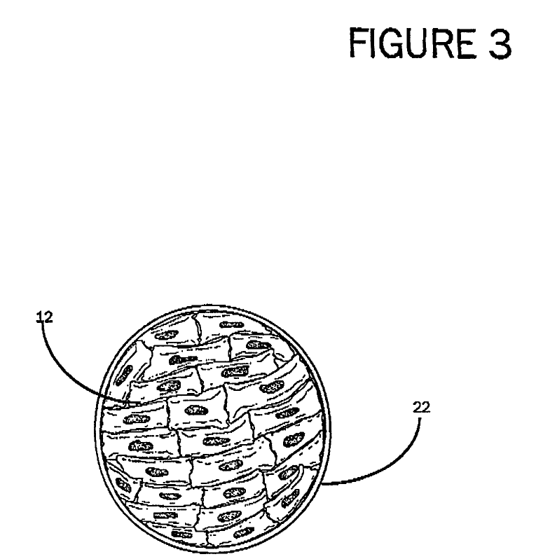

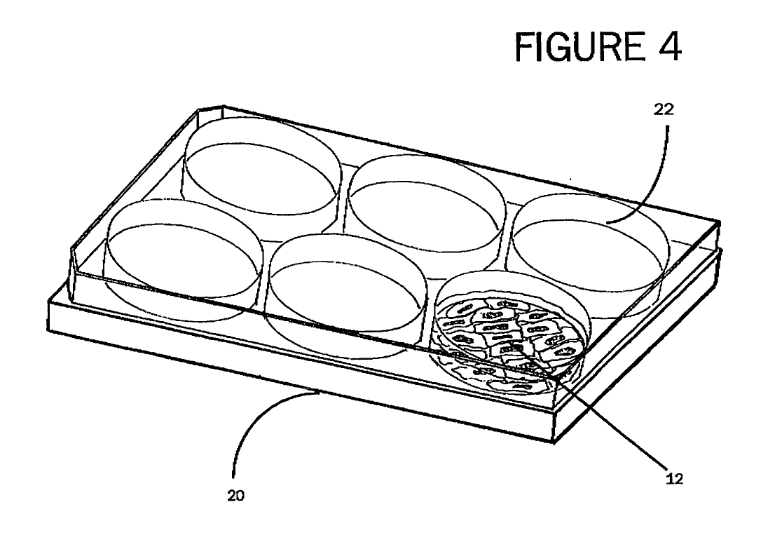

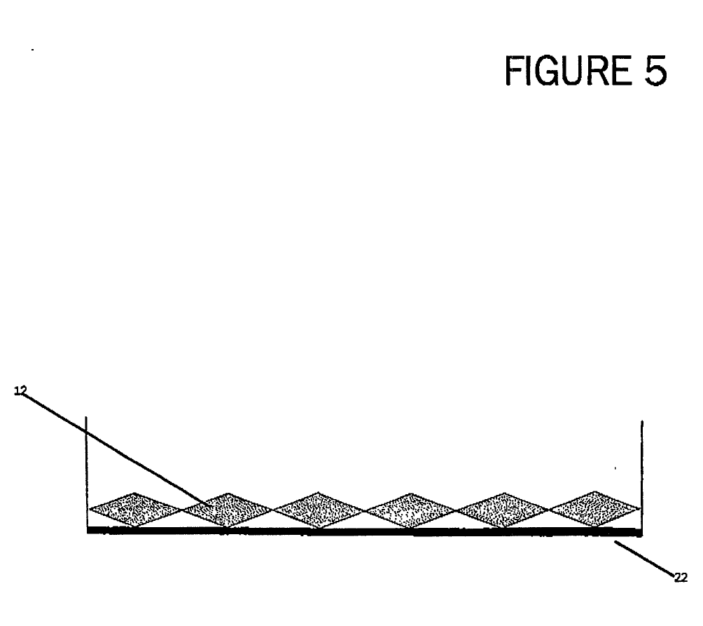

In this regard, FIG. 1 is a flow chart of the preparation of the adherent cells 02 and the tissue culture plate 20. FIG. 2 illustrates the bottom of the well 22 treated with the 1 ml cell suspension 12 solution prior to incubation of the tissue culture plate 20. FIG. 3 illustrates the bottom of the well 22 treated with the 1 ml cell suspension 12 solution after incubation of tissue culture plate 20. FIG. 4 shows a tissue culture plate 20 prior to submersion in liquid nitrogen 26. FIG. 5 shows a side view of a well 22 treated with the adherent cells 02. FIG. 6 shows a tissue culture plate 20, with a foil adhesive cover, after thawing.

When the treated tissue culture plate 20 going to be used, it is sufficient to place it in water at 37° C. and allow it to stand for approximately 4 to 7 minutes. The cryogenic preservative 24 is aspirated from each well 22, followed by a wash step with 500 μl of culture medium 10 per well 22.

The plates then can be used to serve for, but are not limited to, these laboratory functions: They can be used to run various assays, such as epithelial and neutralization assays, and may be used for feeder cell cultures.

Accordingly, it can be seen that the present invention would allow for plates of adherent cells to be frozen at a particular stage of confluency and be ready for use upon thawing.

Although the description above contains many specificities, these should not be construed as limiting the scope of the invention but as merely providing illustrations of some of the presently preferred embodiments of this invention. Various other embodiments and ramifications are possible within its scope. For example, the invention can utilize various adherent cells 02 and various cryopreservatives 24, and various assays and tissue culture experiments can be run using the invention after thawing. The adherent cells 02, cryopreserved in wells 22, using the above stated method on tissue culture plates 20, after storage at −80° C., can be thawed and used in various assays and as feeder cells. After being cryopreserved and stored in the above stated method, the adherent cells 02 maintain both their morphological and functional characteristics, sufficient for their intended use. A number of plant, animal, and insect cells, including but not limited to, HeLa-CD4-LTR-β-gal, F4, B9, F2, epithelial, T cells, lymphoblastoid, H9, C8166, Molt, Molt-4, CEM, Jurkat, and CEM74, can be cryopreserved and stored using the above-stated method.

The invention method utilizes the tissue culture plate 20 wherein the adherent cells 02 are receptive to infection by a plurality of viruses including, but not limited to, HIV, SIV, BIV, and SHIV. The tissue culture plate 20 utilizes the adherent cells 02 listed above as a means for making this invention function. The tissue culture plate 20 also utilizes a foil adhesive cover 28 to enhance storage capabilities and to protect the adherent cells 02 in the wells 22 from contamination. The adhesive foil 28 also protects the wells 22 while allowing even flow and uniform cooling of the cells 02 with liquid nitrogen 26. The method also utilizes a plastic, waterproof, resealable bag 30 for storage and labeling purposes. In order for the invention to obtain consistent volumes of cells 02 per will 22, the invention requires the use of one of two methods of cell counting prior to treatment of the tissue culture plates 20. The first method of cell counting requires the pipetting of 1 ml of cell suspension 12 into a collection vial, placing the vial into a cell counter and reading the digital output, and then diluting the cell suspension 12 until its count reads 4×10⁴ per ml. The data resulting from performing these tests eliminate the necessity for volume and sterility testing to be done later and eliminate varying results while performing other assays and in determining the consumption of feeder cells. The adherent cells 02 are incubated in the wells 22 of the tissue culture plate 20 in a culture medium 10 including DMEM supplemented with 5% heat-inactivated fetal calf serum and 50 units/ml of penicillin/streptomycin. The adherent cells 02 are cryopreserved on a rigid, polystyrene tissue culture plate 20 by treating the adherent cells 02 on a tissue culture plate 20 with a cryopreservant 24. The cryopreservant 24 may be a 10% glycerol or DMSO solution or both, with 30% fetal calf serum. The adhesive foil 28 is placed over the wells 22 on the tissue culture plate 20, and the plate 20 is set into liquid nitrogen 26 at −160° C. and allowed to stand for 1 minute. The tissue culture plate 20 can then be stored in a waterproof, plastic, resealable bag 30 at −80° C. There are various methods of incubation. One method of incubation is to place the tissue culture plate 20 in water at 37° C. for 4 to 7 minutes. The outlined steps eliminate the variability in thawing.

The cryopreserved adherent cells 02 can be stored for at least 3 months. The adherent cells 02, cryopreserved in the tissue culture plate 20 utilizing the above method can be shipped to and utilized by laboratories that do not have the facilities to produce the cultured cells. The invention also provides for more accurate and consistent results arising from the set number of cells 02 per well 22.

### EXAMPLES

### Example 1

The following example further illustrates one preferred embodiment of the method of the present invention, but is neither intended nor should be considered as limitative in scope. Using HeLa-CD4-LTR-β-gal cells 02 in a flask 04, they are incubated in an incubator 06 at 37° C. at 5% CO₂ levels. The flask 04 bearing HeLa-CD4-LTR-β-gal cells 02 is removed from the incubator 06 and aspirated. Next, 5 ml of the culture medium 10 is added to the flask 04, wherein the culture medium is comprised as follows:

RPMI 1640 10% heat-inactivated fetal serum;

10% fetal calf serum;

10 mM gentamicin;

200 mM of L-glutamine; and

non-essential amino acids and vitamins.

Using a pipette, 10 ml of the cell suspension 12 is removed from the flask 04 and placed into a 15 ml. centrifuge tube. The tube is centrifuged for 7 minutes at 1000×g. The cells form pellets at the bottom of the supernatant 32, whereupon the supernatant 32 is aspirated out of the tube and the cell pellets are re-suspended in 10 ml. of the culture medium 10. One droplet from the suspension 12 is removed by a Pasteur pipette, placed on a hemacytometer 16, and covered with a slip. The hemacytometer 16 is placed under a microscope 18 and the cells are counted. Using the cell count, the number of cells 02 per milliter is adjusted by diluting the cell suspension 12 with the culture medium 10 until the cell count reaches 4×10⁴ per ml. Then 1 ml of the cell suspension 12 is pipetted in each well 22 of a rigid styrene plastic well plate 20. The suspension 12 is only permitted to cover up to 30% to 50% of the bottom of the wells. The amount of culture medium per well varies with the size of the tissue culture plate wells and the plurality of wells, e.g., 200λ for a 96-well plate, 500λ for a 48-well plate, 1 ml for a 24-well plate, 3 ml for a 12-well plate, and 5 ml for a 6-well plate. The current embodiment utilizes a 24-well plate. At this point, the plate 20 is incubated for 1 hour, with a culture medium containing 30% fetal calf serum, in the CO₂ incubator 06 at 37° C. with 5% CO₂, allowing cells 02 to grow in the well 22. The medium 10 is then aspirated from each well 22 and the plate 20 is prepared for cryogenic preservation as follows: 100 μl of 10% DMSO solution, with 30% fetal calf serum at 4° C. is pipetted into each well 22. An adhesive foil cover 28 is sealed over the top of the tissue culture plate 20, thus protecting the adherent cells 02, ensuring moveability of the tissue culture plate 20, and permitting movement of the liquid nitrogen 26 around each individual well 22. The tissue culture plate 20 is immersed in a container holding 200 ml of liquid nitrogen 26 at −160° C. The operator is careful not to allow any liquid nitrogen 26 to get into any individual well 22. The tissue culture plate 20 is allowed to stand in the liquid nitrogen 26 for 1 minute. The plate 20 is then placed in a plastic, waterproof, resealable bag 30 and stored at −80° C. To use, the waterproof, resealable bag 30 is allowed to warm to room temperature by placing it into container of water at 37° C. for approximately 4 to 7 minutes. The tissue culture plate 20 is then removed from the bag 30. The adhesive foil 28 is removed to expose the wells 22 for inoculation. The adherent cells 02 are then infected with HIV and used for infectivity testing for HIV.

### Example 2

The following example further illustrates one preferred embodiment of the method of the present invention, but is neither intended nor should be considered as limitative in scope. Using MDCK cells 02 in a flask 04, they are incubated in an incubator 06 at 37° C. at 5% CO₂ levels. The flask 04 bearing MDCK cells 02 is removed from the incubator 06 and aspirated. Next, 5 ml of the culture medium 10 is added to the flask 04, wherein the culture medium is comprised as follows:

RPMI 1640 10% heat-inactivated fetal serum;

5% fetal calf serum;

10 mM gentamicin;

200 mM of L-glutamine; and

non-essential amino acids and vitamins.

Using a pipette, 10 ml of the cell suspension 12 is removed from the flask 04 and placed into a 15 ml centrifuge tube. The tube is centrifuged for 7 minutes at 1000×g. The cells form pellets at the bottom of the supernatant 32 whereupon the supernatant 32 is aspirated out of the tube and the cell pellets are re-suspended in 10 ml of the culture medium 10. One droplet from the suspension 12 is removed by a Pasteur pipette, placed on a hemacytometer 16, and covered with a slip. The hemacytometer 16 is placed under a microscope 18 and the cells are counted. Using the cell count, the number of cells 02 per milliliter is adjusted by diluting the cell suspension 12 with the culture medium 10 until the cell count reaches 4×10⁴ per ml. Then 3 ml of the cell suspension 12 is pipetted in each well 22 of a rigid styrene plastic well plate 20. The suspension 12 is uniformly applied to 100% of the bottom of the wells 22. The current embodiment utilizes a 12-well plate.

At this point, the plate 20 is incubated for 1 hour, with a culture medium containing 30% fetal calf serum, in the CO₂ incubator 06 at 37° C. at 5% CO₂, allowing cells 02 to grow in the well 22. The medium 10 is then aspirated from each well 22 and the plate 20 is prepared for cryogenic preservation as follows: 100 μl of 10% glycerol solution, with 30% fetal calf serum at 4° C., is pipetted into each well 22. An adhesive foil cover 28 is sealed over the top of the tissue culture plate 20, thus protecting the adherent cells 02, ensuring moveability of the tissue culture plate 20, and permitting movement of the liquid nitrogen 26 around each individual well 22. The tissue culture plate 20 is immersed in a container holding 200 ml of liquid nitrogen 26 at −160° C. The operator is careful not to allow any liquid nitrogen 26 to get into any individual well 22. The tissue culture plate 20 is allowed to stand in the liquid nitrogen 26 for 1 minute. The plate 20 is then placed in a plastic, waterproof, resealable bag 30 and stored at −80° C. To use, the waterproof, resealable bag 30 is allowed to warm to room temperature by placing it into container of water at 37° C. for approximately 4 to 7 minutes. The tissue culture plate 20 is then removed from the bag 30. The adhesive foil 28 is removed to expose the wells 22 for inoculation. The adherent cells 02 are then used in viral plaque assays.

Thus the scope of the invention should be determined by the appended claims and their legal equivalents, rather than by the examples given.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited above and in the figures are hereby incorporated by reference.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A cryopreserved cell culture assembly, comprising:
a tissue culture plate having at least one well bounded by a bottom wall surface upon which cells can attach and grow, and a plurality of side walls forming a top rim, said rim defining a well opening;
adherent cells attached to said bottom wall surface, wherein said cells contain an amount of a cryoprotectant which is effective to protect them from damage when stored frozen, wherein said cells are in the frozen state.

2. A cryopreserved cell culture assembly of claim 1, wherein said cells are animal cells or insect cells.

3. A cryopreserved cell culture assembly of claim 2, wherein said cells are mammalian cells.

4. A cryopreserved cell culture assembly of claim 3, wherein said cells are receptive for infection by HIV-1.

5. A cryopreserved cell culture assembly of claim 4, wherein said receptive cells are HeLa-CD4-LTR-β-gal, HeLa-CD4-CCR5-LTR-β-gal, H9, C8166, Molt-4, Jurkat, CEMX174, HUT 78, or U87.CD4.

6. A cryopreserved cell culture assembly of claim 1, wherein the cryoprotectant is dimethylsulfoxide, glycerol, propylene glycol, ethylene glycol, or hydroxyethyl starch.

7. A cryopreserved cell culture assembly of claim 1, wherein said plate comprises six wells.

8. A cryopreserved cell culture assembly of claim 1, wherein said plate comprises twenty-four wells.

9. A cryopreserved cell culture assembly of claim 1, further comprising a cover fit over said rim of said well.

10. A cryopreserved cell culture assembly of claim 9, wherein said cover is an adhesive foil cover.

11. A cryopreserved cell culture assembly of claim 1, wherein said plate is polystyrene.

12. A cryopreserved cell culture assembly of claim 1, wherein said cells are frozen at about −20° C., about −80° C., or about −160° C., depending on the nature of the cells being frozen.

13. A method of preparing a cryopreserved cell culture assembly, comprising:
(a) contacting adherent cells attached to a bottom wall surface of a tissue culture plate with an amount of a cryoprotectant effective to protect said cells from damage when freezing,
said tissue culture plate having at least one well bounded by said bottom wall surface upon which cells can attach and grow, and a plurality of side walls forming a top rim, said rim defining a well opening;
(b) sealing said tissue culture plate by placing a cover over said top rim; and
(c) freezing said adherent cells attached to said bottom wall surface.

14. A method of claim 13, wherein said cells are animal cells or insect cells.

15. A method of claim 14, wherein said cells are mammalian cells.

16. A method of claim 15, wherein said cells are receptive for infection by HIV-1.

17. A method of claim 13, wherein the cryoprotectant is dimethylsulfoxide, glycerol, propylene glycol, ethylene glycol, or hydroxyethyl starch.

18. A method of claim 13, wherein said cover is an adhesive foil cover.

19. A method of claim 13, wherein said plate is polystyrene.

20. A method of claim 13, wherein said freezing is at a temperature of about −20° C., about −80° C., or about −160° C., depending on the cells being frozen.
